(19) United States
(12) Patent Application Publication
(10) Pub. No.: US 2010/0143471 A1
(43) Pub. Date: Jun. 10, 2010

(54) NOVEL REDUCED DOSE PHARMACEUTICAL COMPOSITIONS OF FEXOFENADINE AND PSEUDOEPHEDRINE

(75) Inventors: Ubaidulla Udhumansha, Pune, Maharashtra (IN); Shirishkumar Kulkarni, Maharashtra (IN)

Correspondence Address: MERCHANT & GOULD PC, P.O. BOX 2903, MINNEAPOLIS, MN, 55402-0903, US

(73) Assignee: LUPIN LIMITED, MAHARASHIRA (IN)

(21) Appl. No.: 12/528,405

(22) PCT Filed: Mar. 17, 2008
(86) PCT No.: PCT/IN08/00156

§ 371 Date: Jan. 18, 2010

(30) Foreign Application Priority Data
Mar. 21, 2007 (IN) 430/KOL/2007

(51) Int. Cl. A61K 9/24; A61K 31/445; A61P 11/02; A61K 9/00; A61K 9/16; A61P 37/08

(52) U.S. Cl. 424/472; 514/317; 424/484; 424/490

(57) ABSTRACT

A novel reduced dose pharmaceutical composition comprising combination of fexofenadine or salts thereof; and pseudoephedrine or salts thereof in therapeutically effective amount, for the treatment of allergic rhinitis and associated symptoms in pediatric population.

### FIELD OF THE INVENTION

The present invention relates to novel reduced dose pharmaceutical compositions comprising therapeutically effective amount of fexofenadine or salts thereof and psuedoephedrine or salts thereof.

### BACKGROUND OF THE INVENTION

Antihistaminics and decongestants act by different mechanism to treat allergic reactions. Decongestants constrict vessels in the nasal mucus membranes and thereby decrease tissue swelling and nasal congestion. Decongestants are found to be better than antihistamines for restoring the potency of congested nasal airways. Histamine is a mediator released from cells, which line the walls of the nasal mucous membranes (mast cells). When released, histamine binds to local histamine receptors, thereby causing sneezing, nasal itching, swelling of the nasal membranes, and increased nasal secretions.

Antihistamines relieve these effects, albeit by a different mechanism than that of decongestants.

Antihistamines block the binding of histamines to the histamine receptors by preoccupying the histaminic receptors. Consequently they are effective only if given prior to histamine release since once histamine is released and binds to the receptors, it is too late. Although individuals typically take antihistamines after symptoms occur, it is more desirable to dose antihistamines so as to effect therapeutic availability in anticipation of histamine release. Combining antihistamines and decongestants utilizes both mechanistic approaches, and has been shown to offer more complete relief of allergic symptoms than therapy with either component alone.

U.S. Pat. No. 6,267,986 B1 describes a process for the preparation of a controlled release pharmaceutical composition comprising two discrete zones wherein the first discrete zone comprises therapeutically effective amount of Pseudoephedrine or its pharmaceutically acceptable salts as active ingredient and the second discrete zone comprises a therapeutically effective amount of a long-acting antihistamine selected from the group consisting of Loratadine, Azatidine, Fexofenadine, Terfenadine, Cetirizine, Astemizole, and Levocabastine, or their pharmaceutically acceptable salts as active ingredient.

U.S. Pat. No. 4,996,061 discloses a pharmaceutical composition in the form of a multiple-compression tablet comprising a discrete zone made from a formulation which provides sustained-release of a therapeutically effective decongestant amount of a sympathomimetic drug and a discrete zone made from a different formulation which provides immediate release of a therapeutically effective antihistaminic amount of a piperidinoalkanol and, optionally, a therapeutically effective decongestant amount of a sympathomimetic drug.

U.S. Pat. No. 6,039,974 describes pharmaceutical composition in the form of a bilayer tablet comprising (a) a first discrete zone made up with a therapeutically effective decongestant amount of a sympathomimetic drug or its salts with carnauba wax and a suitable antiadherent, which provides sustained release of the sympathomimetic drug. The second discrete zone made up of piperidinoalkanol or a pharmaceutically acceptable salts in a second carrier base material comprising mixture of cellulose diluents, binder, disintegrant and lubricants in preferred concentrations.

U.S. Pat. No. 6,039,974 further discloses the therapeutically effective decongestant amount of a sympathomimetic drug varying from about 1 mg to about 200 mg. Preferred amounts will vary from about 5 mg to about 150 mg, with about 120 mg administered twice daily being most preferred. The patent also discloses the therapeutically effective antihistaminic amount of a piperidinoalkanol compound varying over a wide range from about 0.1 mg to about 240 mg. The preferred therapeutically effective antihistaminic amount of a piperidinoalkanol compound will vary from about 20 mg to about 70 mg with about 60 mg administered twice daily being most preferred.

US Pat. Application 2006/0182800 relates to pharmaceutical composition for antihistaminic-decongestant combination in the form of unit dosage form, specifically the invention relates to bi-layered tablet formulation

U.S. Pat. No. 6,613,357 discloses an osmotic device containing pseudoephedrine and an H1 antagonist, or antihistamine. More particularly, it pertains to an osmotic device tablet, which provides a controlled release of pseudoephedrine and a rapid or immediate release of an H1 antagonist.

U.S. Pat. RE39, 069 and U.S. Pat. No. 6,004,582 pertains to an osmotic device for the controlled delivery of active agents to an environment of use. More particularly, it pertains to a multi-layered osmotic device that allows the immediate delivery of a first active agent followed by a monitored, continuous, controlled and/or retarded delivery of a second active agent which is the same or different as the first active agent.

Safety and effectiveness of marketed product ALLEGRA-D 24 HOUR and ALLEGRA-D 12 HOUR in children below the age of 12 years have not been established. In addition, the doses of the individual components in ALLEGRA-D 24 HOUR exceed the recommended individual doses for pediatric patients under 12 years of age. ALLEGRA-D 24 HOUR is not recommended for pediatric patients under 12 years of age.

Wahn et. al disclose fexofenadine, at doses of up to 60 mg bid is safe and nonsedating and fexofenadine HCl 30 mg bid effectively reduces all seasonal allergic rhinitis symptoms in children aged 6-11 years.

Although each of the patent and patent applications represents a combination of antihistaminics and decongestants in general, there still exists a need to provide delivery systems to enable safe, convenient and effective dosing of active pharmaceutical ingredients to pediatric patients. Moreover, the need of pediatric formulation development comprises minimal dosing frequency and minimum impact on life style along with convenient, easy and reliable method of administration.

Thus, the present invention provides novel reduced dose pharmaceutical compositions comprising fexofenadine or salts thereof and pseudoephedrine or salts thereof for pediatric patients.

### OBJECTS OF THE INVENTION

The objective of the present invention is to provide novel reduced dose pharmaceutical compositions comprising fexofenadine or salts thereof and pseudoephedrine or salts thereof for the treatment of allergic rhinitis in pediatric population.

Another object of present invention is to provide a pharmaceutical formulation at reduced doses of fexofenadine or salts thereof in the range of 10 mg to 60 mg and psuedoephedrine or salts thereof in the range of 20 mg to 120 mg for pediatric population.

Yet another object of the present invention is to provide a pharmaceutical formulation at reduced dose comprising 30 mg of fexofenadine or salts and 60 mg of psuedoephedrine or salts thereof for pediatric population.

Yet another object of the present invention is to provide reduced dose pharmaceutical compositions, which comprises fexofenadine or salts thereof in immediate release form and pseudoephedrine or salts thereof in controlled release form.

Yet another objective of the present invention is to provide reduced dose pharmaceutical compositions, wherein pharmaceutical compositions are in the form of solid oral dosage forms.

Yet another objective of the present invention is to provide reduced dose pharmaceutical compositions, wherein a pharmaceutical composition is in the form of liquid oral dosage forms.

Yet another object of present invention is to provide a kit, wherein the kit comprises: (a) an oral dosage form comprising a therapeutically reduced dose of fexofenadine or salts thereof (b) an oral dosage form comprising a therapeutically reduced dose of pseudoephedrine or salts thereof: in a sachet or in a suitable container and (c) instructions to reconstitute with liquids.

Yet another object of present invention is to provide a kit, wherein the kit comprises: (a) an oral solid dosage forms comprising a therapeutically reduced dose of fexofenadine or salts thereof (b) an oral solid dosage forms comprising a therapeutically reduced dose of pseudoephedrine or salts thereof by packaging on separate sheets. Yet another object of the present invention is to provide a reduced dose pharmaceutical compositions comprising fexofenadine or salts thereof and pseudoephedrine or salts thereof, in which the compositions exhibits in vitro release of fexofenadine in 0.1 N HCl of not less than about 80% after 2 hours, and/or in vitro release of pseudoephedrine or salts thereof in 0.001 N HCl of not less than about 80% after 22 hours.

Preferably, the fraction of pseudoephedrine or salts thereof that is released is not less than about 80% after 15 hours, especially not less than about 80% after 12 hours.

### SUMMARY OF THE INVENTION

According to one aspect of the present invention there is provided novel reduced dose pharmaceutical compositions comprising fexofenadine or salts thereof and pseudoephedrine or salts thereof for the treatment of allergic rhinitis in pediatric population.

According to another aspect of present invention there is provided a pharmaceutical formulation at reduced doses of fexofenadine or salts thereof in the range of 10 mg to 60 mg and psuedoephedrine or salts thereof in the range of 20 mg to 120 mg for pediatric population.

According to another aspect of present invention there is provided a pharmaceutical formulation at reduced doses comprising 30 mg of fexofenadine or salts and 60 mg of psuedoephedrine or salts thereof for pediatric population.

A further aspect of the present invention is to provide reduced dose pharmaceutical compositions, which comprises fexofenadine or salts thereof in immediate release form and pseudoephedrine or salts thereof in controlled release form.

Yet another aspect of the present invention is to provide reduced dose pharmaceutical compositions, wherein pharmaceutical compositions are in the form of solid oral dosage forms.

A further aspect of the present invention is to provide reduced dose pharmaceutical compositions, wherein a pharmaceutical composition is in the form of liquid oral dosage forms.

Yet another aspect of present invention is to provide a kit, wherein the kit comprises: (a) an oral dosage form comprising a therapeutically reduced dose of fexofenadine or salts thereof (b) an oral dosage form comprising a therapeutically reduced dose of pseudoephedrine or salts thereof: in a sachet or in a suitable container and (c) instructions to reconstitute with liquids.

Another aspect of present invention is to provide a kit, wherein the kit comprises: (a) an oral solid dosage forms comprising a therapeutically reduced dose of fexofenadine or salts thereof (b) an oral solid dosage forms comprising a therapeutically reduced dose of pseudoephedrine or salts thereof by packaging on separate sheets.

According to another aspect of the present invention there is provided a reduced dose pharmaceutical compositions comprising fexofenadine or salts thereof and pseudoephedrine or salts thereof, in which the compositions exhibits in vitro release of fexofenadine in 0.1 N HCl of not less than about 80% after 2 hours, and/or in vitro release of pseudoephedrine or salts thereof in 0.001 N HCl of not less than about 80% after 22 hours.

### BRIEF DESCRIPTION OF THE DRAWINGS

FIG. 1(a) shows a release profile of Fexofenadine of Example 1 in Type II USP apparatus, 0.1N HCl, 900 ml, and 50 rpm.

FIG. 1(b) shows a release profile of Pseudoephedrine of Example 1 in Type II USP apparatus 0.001N HCl, 900 ml, and 50 rpm.

FIG. 2(a) shows a release profile of Fexofenadine of Example 2 in Type II USP apparatus, 0.1N HCl, 900 ml, and 50 rpm.

FIG. 2(b) shows a release profile Pseudoephedrine of Example 2 in Type II USP apparatus 0.001N HCl, 900 ml, and 50 rpm.

### DETAILED DESCRIPTION OF THE INVENTION

The use of fexofenadine and psuedoephedrine in their free base, free acid, racemic, optically pure, diastereomeric and/or pharmaceutically acceptable salts forms is well recognized by those skilled in the art as safe and effective antiallergic and nasal decongestant respectively.

It is well known to those skilled in the art that controlled delivery results in a decrease in the frequency of drug administration thereby improving patient compliance. Furthermore, controlled drug delivery systems produce constant therapeutic plasma levels of active ingredients as compared to fluctuations seen when multiple doses of a conventional formulation are given. Thus, controlled drug delivery systems may decrease the severity and frequency of side effects.

The term “controlled release” as used herein in relation to the composition according to the invention or a rate controlling agent or used in any other context means release, which is not immediate release and is taken to encompass sustained release, prolonged release, timed release, retarded release and extended release.

A decongestant is commonly administered orally in combination with an antihistamine for relieving nasal congestion associated with allergic rhinitis. It is quite apparent from the above stated facts that when the decongestant is pseudoephedrine or salts thereof or its pharmaceutically acceptable salts, and the antihistamine is a long-acting antihistamine that is fexofenadine or salts thereof, then the dosage form should preferably be designed such that the fexofenadine is released in a conventional manner and pseudoephedrine is released at a controlled rate such that the pharmaceutical composition is suitable for twice-daily or once-daily administration.

A therapeutically effective reduced dose antihistaminic amount of fexofenadine or salts thereof may vary over a wide range is from about 0.1 mg to about 60 mg. The preferred therapeutically effective reduced dose antihistaminic amount of a fexofenadine will vary from about 10 mg to about 60 mg with about 30 mg administered twice daily being most preferred.

Alternatively in the pediatric population the pharmaceutical composition comprising 60 mg of fexofenadine or salts thereof and 120 mg of pseudoephedrine or salts thereof can be administered in once daily dosage.

The present invention comprises therapeutically effective reduced dose decongestant amount of pseudoephedrine in pharmaceutical formulation to provide a prolonged or sustained release of pseudoephedrine whereas fexofenadine is in an immediate release form. As used herein the term “immediate release” refers to a property of the pharmaceutical composition wherein the entire dose of fexofenadine is made bioavailable without substantial delay.

The term pediatric population refers to patients in need of an antihistamine, antiallergy agent, bronchodilator or requires treatment of urticaria in the range of age group from 3-12 years. The most preferred age group being from 6-12 years.

The excipients for enabling the sustained release of pseudoephedrine or salts thereof comprise hydrophilic polymers and/or hydrophobic polymers.

The hydrophilic release controlling agents are selected from but are not limited to hydroxypropyl methylcellulose (HPMC), hydroxypropyl cellulose (HPC), polyethylene oxide, polyvinyl alcohol, polyvinylpyrrolidone, xanthan gum, guar gum, chitosan and its derivatives, carbomer, carrageenan, carboxymethyl cellulose, sodium alginate, polyglycolized glycerides, polyethylenglycol, or mixture thereof.

The hydrophobic release controlling agents are selected from but are not limited to ammonium methacrylate copolymers type A and B as described in USP, methacrylic acid copolymer type A, B and C as described in USP, polyacrylate dispersion 30% as described in Ph. Eur., polyvinyl acetate dispersion, ethylcellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, carnauba wax, paraffin wax, microcrystalline wax, and. ozokerite; fatty alcohols such as cetostearyl alcohol, stearyl alcohol, cetyl alcohol and myristyl alcohol, and fatty acid esters such as glyceryl monostearate; glycerol monooleate, acetylated monoglycerides, tristearin, tripalmitin, cetyl esters wax, glyceryl palmitostearate, glyceryl behenate, and hydrogenated vegetable oils.

In a preferred embodiment for the preparation of sustained release part of formulation hydrophilic polymers comprises polyglycolized glycerides (Gelucire), and polyethylene glycol.

Yet in the most preferred embodiment polyglycolized glycerides (Gelucire) 50/13 and/or polyethyleneglycol (PEG 2000-20000) is used in the preparation for the sustained release part of formulation.

In another preferred embodiment, for the preparation of sustained release part of formulation hydrophobic agents comprises ethylcellulose, hydrogenated vegetable oils and waxes.

Yet in the most preferred embodiment hydrogenated vegetable oils are used in the preparation for the sustained release part of formulation.

The pseudoephedrine part of the said formulation may further comprise of suitable diluents, binders and lubricants.

The immediate release part of said formulation comprises of fexofenadine, its salts and derivatives thereof along with suitable diluents, binders, disintegrants and lubricants known in the art.

The process of preparation of sustained release part of the formulation comprises but not limited to spray drying, melt granulation, spray coating, hot melt coating, spray congealing and solvent evaporation.

The process of preparation of immediate release part of formulation comprises but not limited to wet granulation, dry granulation and direct compression.

The formulation of the present invention is not restricted to any particular type of formulation. Thus various types of controlled or sustained release type formulations may be used for embodying the present invention, such as for example coated beads, gel matrix tablets, osmotic tablets, multilayer tablets, multicoated tablets, liquid oral and suspension dosage forms etc.

In another embodiment of the present invention the pharmaceutical composition exhibits in vitro release of the fexofenadine in 0.1 N HCl of not less than about 80% after 2 hours, and/or in vitro release of pseudoephedrine or salts thereof in 0.001 N HCl of not less than about 80% after 22 hours.

Preferably the in vitro release of the pseudoephedrine fraction of the pharmaceutical composition is not less than about 80% after 15 hours, especially not less than about 80% after 12 hours.

More Preferably the in vitro release of the pseudoephedrine fraction of the pharmaceutical composition after 7 hours is not less than about 50%, especially not less than about 80%.

The dissolution conditions employed for measuring the release rate utilizes the United States Pharmacopoeia (USP) Apparatus II (Paddle) at 50 rpm with 900 ml of 0.1N HCl at 37.degree. C. for Fexofenadine and United States Pharmacopoeia (USP) Apparatus II (Paddle) at 50 rpm with 900 ml of 0.001 N HCl at 37.degree. C. for Pseudoephedrine.

In another embodiment of the present invention both Pseudoephedrine sustained release composition and fexofenadine immediate release composition are packed in a kit, wherein the kit comprises: (a) an oral dosage form containing a therapeutically reduced amount of fexofenadine (b) an oral dosage form containing a therapeutically reduced amount of pseudoephedrine: in a sachet or in a suitable container and (c) instructions to reconstitute with liquids and administer the fexofenadine and pseudoephedrine.

Alternatively both Pseudoephedrine sustained release composition and fexofenadine immediate release composition are packed in a kit, wherein the kit comprises: (a) an oral solid dosage forms comprising a therapeutically reduced amount of fexofenadine or salts thereof (b) an oral solid dosage forms comprising a therapeutically reduced amount of pseudoephedrine or salts thereof by packaging on separate sheets. It is convenient for the use by patients, however, to prepare kits by packaging fexofenadine dosage form on one side and a pseudoephedrine dosage form on the other side of one and the same sheet (e.g. press through package (PTP) sheet or strip packaging sheet), or by packaging the doses of both preparations for one course of treatment, for instance, on one and the same sheet.

The invention is now described by following non-limiting illustrative examples:

### Example 1

| | | |
|---|---|---|
| S. No | Ingredients | Quantity (mg/tablet) |

Pseudoephedrine HCL Sustained Release layer

| | | |
|---|---|---|
| 01 | Pseudoephedrine HCL | 60 |
| 02 | Hydrogenated Vegetable Oil | 80-120 |
| 03 | Magnesium Stearate | 2-5 |

Fexofenadine HCL Immediate Release layer

| | | |
|---|---|---|
| 04 | Fexofenadine HCL | 30 |
| 05 | Microcrystalline cellulose | 30-50 |
| 06 | Lactose Monohydrate | 20-30 |
| 07 | Pregelatinized Starch | 5-15 |
| 08 | Sodium Starch Glycolate | 8-15 |
| 09 | Magnesium stearate | 2-3 |
| 10 | Colloidal silicon dioxide | 1-2.5 |
| 11 | Colorants | 2 |

Coating (Optional)

| | | |
|---|---|---|
| 12 | Film coating | 50 |

### Brief Manufacturing Procedure:

### A. Preparation of Pseudoephedrine HCL Sustained Release Composition

The Hydrogenated Vegetable Oil material is heated up to 60° C. and then pseudoephedrine HCL is slowly added to the melted wax with continuous stirring. The temperature of the melt is maintained at 60° C. for 30 min. After cooling to room temperature the hard mass was obtained. The mass was passed through suitable sieve. The final granules were kept in well-closed container.

### B. Preparation of Fexofenadine HCL Immediate Release Composition

Blend Fexofenadine HCL, Microcrystalline cellulose, Lactose Monohydrate, Pregelatinized Starch, Sodium Starch Glycolate, Magnesium Stearate and Colloidal Silicon Dioxide. Slug this blend and then sift using sieve of suitable mesh size. Repeat the process to get uniform granules of desired yield. Lubricate the granules with colloidal silicon dioxide and magnesium stearate.

Granules thus obtained can be used for the preparation of pharmaceutical formulation in the form of solid and liquid oral dosage forms.

Solid dosage forms include but not limited to capsules, tablets, multilayer tablets, osmotic tablets, gel matrix, coated beads and multicoated tablets.

The capsules can be opened and all of the contents sprinkled onto a teaspoon of soft food such as applesauce or pudding, which does not require chewing. This mixture should be swallowed immediately and not chewed.

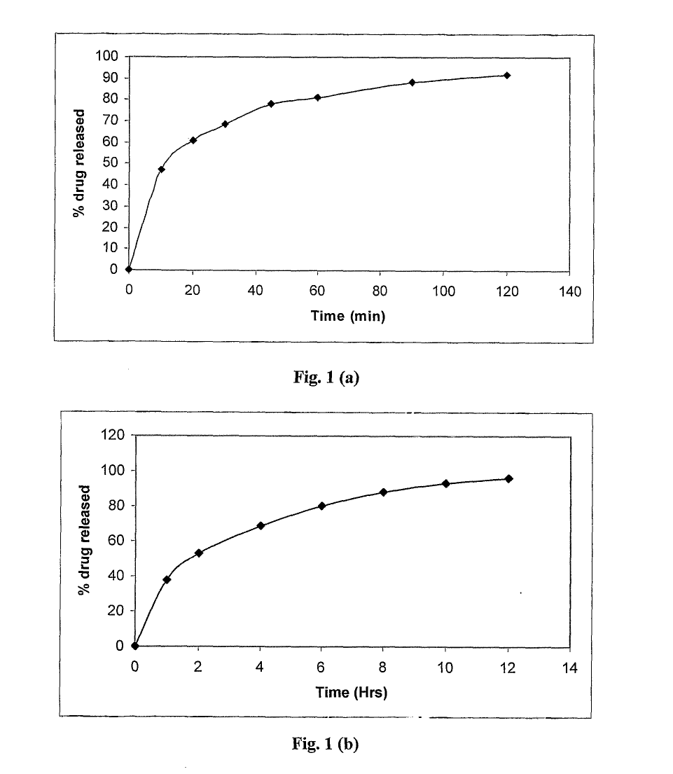

The dissolution data in FIGS. 1(a) and 1 (b) are of fexofenadine and Pseudoephedrine when the granules are compressed into a bilayer tablet dosage form.

### Example 2

| | | |
|---|---|---|
| S. No | Ingredients | Quantity/bottle |

Pseudoephedrine HCL Sustained Release layer

| | | | |
|---|---|---|---|
| 01 | Pseudoephedrine HCl | 60 | mg |
| 02 | Hydrogenated Vegetable Oil | 80-120 | mg |
| 03 | Magnesium Stearate | 2-5 | mg |

Fexofenadine HCL Immediate Release layer

| | | | |
|---|---|---|---|
| 04 | Fexofenadine HCl | 30 | mg |
| 05 | Hydroxy propyl methyl cellulose | 10-20 | mg |
| 06 | Sucrose | 15 | gm |
| 09 | Hydroxy Propyl Cellulose | 0.015 | gm |
| 10 | Xanthan gum | 0.015 | gm |
| 11 | Titanium Dioxide | 0.100 | gm |
| 12 | Flavours | 0.070 | gm |
| 13 | Colloidal Silicon Dioxide | 0.100 | gm |

### Brief Manufacturing Procedure

The wax material is heated up to 60° C. and then pseudoephedrine HCL is slowly added to the melted wax with continuous stirring. The temperature of the melt is maintained at 60° C. for 30 min. After cooling to room temperature the hard mass was obtained. The mass was passed through sieve # 18. The final granules were kept in well-closed container. Fexofenadine HCl dissolved in Hydroxypropyl methylcellulose solution and coated on to the pseudoephedrine granules. The resultant granules were mixed with excipient no's 06-13.

Liquid dosage forms include but are not limited to suspension, solution.

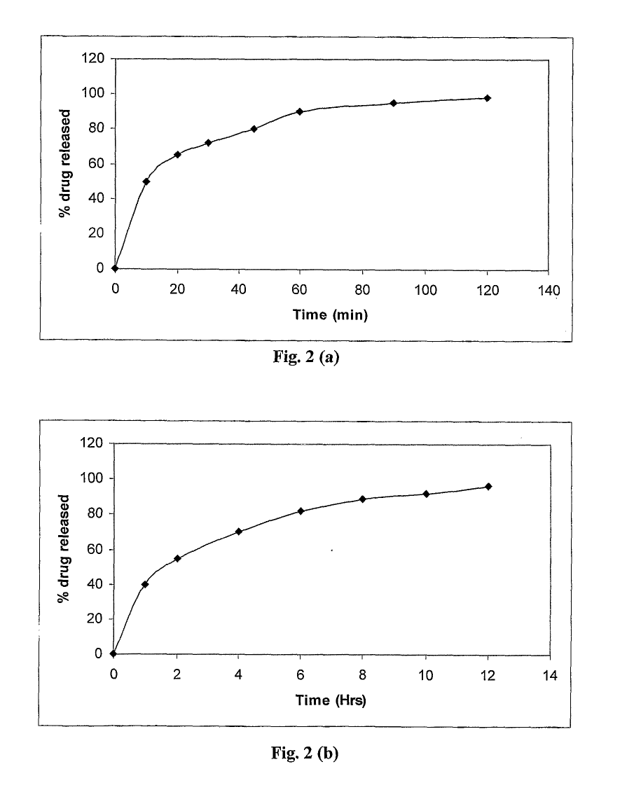

The dissolution data in FIGS. 2(a) and 2 (b) are of Fexofenadine and Pseudoephedrine when the granules are reconstituted with water.

1. A novel reduced dose pharmaceutical composition comprising combination of fexofenadine or salts thereof; and pseudoephedrine or salts thereof in therapeutically effective amount, for the treatment of allergic rhinitis and associated symptoms in pediatric population.

2. A novel reduced dose pharmaceutical composition comprises combination of fexofenadine or salts thereof; and pseudoephedrine or salts thereof in therapeutically effective amount, wherein therapeutically effective amount of fexofenadine or salts thereof is in the range of 10 mg to 60 mg and psuedoephedrine or salts thereof in the range of 20 mg to 120 mg for pediatric population.

3. A novel reduced dose pharmaceutical composition comprises combination of fexofenadine or salts thereof; and pseudoephedrine or salts thereof in therapeutically effective amount, wherein the therapeutically effective amount of fexofenadine or salts thereof is 30 mg and psuedoephedrine or salts thereof is 60 mg for pediatric population.

4. A pharmaceutical composition according to claim 1, wherein fexofenadine is in immediate release form and psuedoephedrine is in controlled release form.

5. A pharmaceutical composition according to claim 4, is a solid oral dosage forms.

6. A pharmaceutical composition according to claim 5 wherein the solid oral dosage form is selected from capsules, tablets, multilayer tablets, osmotic tablets, gel matrix, coated beads and multicoated tablets.

7. A pharmaceutical composition according to claim 6 in the form of a capsule comprising fexofenadine or salts thereof in immediate release form and pseudoephedrine or salts thereof in controlled release form.

8. A pharmaceutical composition according to claim 7 wherein the immediate release form comprising fexofenadine is in the form of powder, granules or pellets.

9. A novel reduced dose pharmaceutical composition of claim 4 wherein the controlled release of pseudoephedrine or salts thereof is achieved by a rate-controlling agent.

10. A pharmaceutical composition of claim 9 wherein the rate controlling agent is selected from the group comprising hydrophilic polymers, hydrophobic polymers, hydrogenated vegetable oils, waxes or combinations thereof.

11. A novel reduced dose pharmaceutical composition comprising a kit; wherein the kit comprises: (a) an oral solid dosage forms comprising a therapeutic amount of fexofenadine or salts thereof (b) an oral solid dosage forms comprising a therapeutic amount of pseudoephedrine or salts thereof wherein fexofenadine and pseudoephedrine are packed on separate sheets or by packaging fexofenadine on one side and a pseudoephedrine on the other side of one and the same sheet or by packaging the doses of both preparations for one course of treatment.

12. A novel reduced dose pharmaceutical composition comprises combination of fexofenadine or salts thereof; and pseudoephedrine or salts thereof in therapeutically effective amount, wherein the therapeutically effective amount of fexofenadine or salts thereof is 30 mg and psuedoephedrine or salts thereof is 60 mg for pediatric population is a liquid oral dosage form.

13. A pharmaceutical composition according to claim 12 is a suspension dosage form.

14. A pharmaceutical composition according to claim 12 is a kit, wherein the kit comprises: (a) an oral dosage form comprising a therapeutic amount of fexofenadine or salts thereof (b) an oral dosage form comprising a therapeutic amount of pseudoephedrine or salts thereof: in a sachet or in a suitable container and (c) instructions to reconstitute with liquids.

15. A novel reduced dose pharmaceutical composition comprising combination of fexofenadine or salts thereof; and pseudoephedrine or salts thereof in therapeutically effective amount, wherein said composition exhibits in vitro release of the fexofenadine in 0.1 N HCl of not less than about 80% after 2 hours, and/or in vitro release of pseudoephedrine or salts thereof in 0.001 N HCl of not less than about 80% after 22 hours.

16. A pharmaceutical composition according to claim 15 wherein pseudoephedrine fraction that is released in vitro is not less than about 80% after 15 hours, especially not less than about 80% after 12 hours.

17. A pharmaceutical composition according to claim 15 wherein pseudoephedrine fraction that is released in vitro after 7 hours is not less than about 50%, especially not less than about 80%.
